(19) United States
(12) Patent Application Publication
(10) Pub. No.: US 2009/0318509 A1
(43) Pub. Date: Dec. 24, 2009

(54) Oxime Derivatives as Inhibitors of Macrophage Migration Inhibitory Factor

(76) Inventor: Yousef Al-Abed, Locust Valley, NY (US)

Correspondence Address: AMSTER, ROTHSTEIN & EBENSTEIN LLP, 90 PARK AVENUE, NEW YORK, NY, 10016, US

(21) Appl. No.: 12/227,777

(22) PCT Filed: Jun. 4, 2007
(86) PCT No.: PCT/US07/13139

§ 371 Date: Feb. 10, 2009

Related U.S. Application Data
(60) Provisional application No. 60/811,258, filed on Jun. 5, 2006.

(51) Int. Cl. A61K 31/4406; C07D 213/80; C07C 69/76; C07C 69/74; A61K 31/4409; A61K 31/24; A61K 31/215; A61P 37/02

(52) U.S. Cl. 514/356; 546/322; 560/110; 560/1; 514/534; 514/529; 514/354; 546/326

(57) ABSTRACT

Provided are compounds of formula (I) and other compounds. Also provided are pharmaceutical compositions comprising these compounds. Additionally, methods of inhibiting macrophage migration inhibitory factor (MIF) activity in a mammal are provided, as are methods of treating or preventing inflammation in a mammal, and methods of treating a mammal having sepsis, septicemia, and/or endotoxic shock.

### CROSS-REFERENCE TO RELATED APPLICATION

This application claims the benefit of U.S. Provisional Application No. 60/811,258 filed Jun. 5, 2006.

### BACKGROUND OF THE INVENTION

(I) Field of the Invention

The present invention generally relates to cytokine inhibitors. More specifically, the invention is directed to inhibitors of macrophage migration inhibitory factor.

(2) Description of the Related Art

Sepsis, a potentially lethal systemic inflammatory reaction to infection, affects approximately 700,000 individuals and kills more than 215,000 people annually at a cost of $16.7 billion nationally (Martin et al., 2003). While the incidence of sepsis continues to rise (O'Brien and Abraham, 2003), to date, no small molecule therapeutic agent is currently approved by the FDA for its clinical management. Thus, severe sepsis is a common, expensive, and frequently fatal condition, with as many deaths annually as those from acute myocardial infarction (Angus et al., 2001).

Sepsis is mediated, at least in part, by soluble factors. Among these, macrophage migration inhibitory factor (MIF) has been shown to play a critical role in inflammation pathways. The biology of MIF places it in the macrophage-derived pathways of proinflammatory responses (Bridhuizen et al., 2001; Lue et al., 2002; Calandra, 2000; 2001). MIF was first described in the early 1960s, as a product of activated lymphocytes that inhibited the random movement or migration of cultured monocytes/macrophages (George and Vaughan, 1962; Bloom and Bennett, 1966; David, 1966). This discovery engendered significant interest, as MIF was one of the first soluble, non-immunoglobulin factors that was amenable to study in vitro.

MIF, produced by numerous cell types, including immune and endocrine cells, is now recognized as a pro-inflammatory counter-regulator of the anti-inflammatory activities of the glucocorticoids. In vitro, MIF expression abrogates the anti-inflammatory and immunosuppressive effect of glucocorticoid production on pro-inflammatory cytokines (TNF-α, IL-1, IL-2, IL-6, and IL-8) (Calandra and Bucala, 1997; Donnelly et al., 1997). In mice, administration of recombinant MIF, together with dexamethasone, completely blocks the protective effects of dexamethasone on LPS lethality (Calandra, 1995). MIF is critically involved in the pathogenesis of a variety of inflammatory diseases. In particular, animal models of Gram-positive, Gram-negative, and polymicrobial sepsis, as well as MIF knockout models, indicate a critical role of MIF in sepsis (Calandra et al., 2000; Bozza et al., 1999; Bernhagen et al., 1993). Thus, the numerous pro-inflammatory effects of MIF together with its unique ability to override or counter-regulate the normal physiological inhibition of immune cell activation and pro-inflammatory cytokine cascades by glucocorticoids, position MIF as a critical mediator of sepsis.

In vivo studies demonstrate that MIF is an important late-acting mediator of systemic inflammation. Deletion of the MIF gene in mice conferred protection against lethal endotoxemia staphylococcal toxic shock (Bozza et al., 1999). In addition, administration of neutralizing MIF-antibody protected mice from: (a) LPS-induced lethality; (b) lethal peritonitis and septic shock induced by E. coli peritonitis and (c) fulminant septic shock induced by cecal ligation and puncture (CLP) in TNF-α deficient mice (Calandra, 2001; Bernhagen et al., 1993). In contrast to early mediators such as TNF-α and IL-1β, MIF release peaks and then plateaus 5 hours after the onset of CLP, thereby offering a window of opportunity for therapeutic treatment. Consequently, anti-MIF therapies are potentially more beneficial than anti-TNF-α and anti-IL-1 therapies, which have demonstrated limited benefits for patients with severe sepsis (Abraham, 1999). In contrast, administration of anti-MIF antibody 8 hours post-induction of sepsis confers significant protection in a murine CLP model of sepsis versus animals receiving control IgG. Human studies also support a role for MIF in septic shock (Beishuizen et al., 2001; Calandra et al., 2000). A correlation has been documented between the severity of injury or infection in trauma patients and MIF levels in the serum, with increased circulating levels of M IF displayed in patients with severe sepsis (6-fold) and in patients with septic shock (15-fold) (Calandra et al., 2000). Taken together, these results suggest that an MIF antagonist will prove to be a potent anti-inflammatory agent, acting both by neutralizing the direct inflammatory activity of MIF and by restoring the anti-inflammatory benefits of endogenous or administered corticosteroids.

Three-dimensional X-ray crystallographic studies have shown that MIF appears as a homotrimer (Suzuki et al., 1994; Taylor et al., 1999, Sugimoto et al., 1995; Kato et al., 1996; Lolis and Bucala, 1996; Sugimoto et al., 1996; Sun et al., 1996; Suzuki et al., 1996; Lubetsky et al., 1999; Orita et al., 2001; Lubetsky et al., 2002). MIF possesses the unusual ability to catalyze the tautomerization of D,L-dopachrome methyl esters into their corresponding indole derivatives (Rosengren et al., 1996). More recently, phenylpyruvic acid and p-hydroxyphenylpyruvic acid were found to be MIF substrates (Matsunaga et al., 1999a; Rosengren et al., 1997; Matsunaga et al., 1999b). The crystal structures of MIF complexed with p-hydroxyphenylpyruvic acid has identified an active site which lies in a hydrophobic cavity that forms between two adjacent subunits of the homotrimer (Lubetsky et al., 1999). Proline (Pro-1 of the active site) appears to be the critical residue for enzymatic activity, since site-directed mutagenesis that substitutes a serine (P1-s) or glycine (P1-g) for Pro-1 results in mutants devoid of D-dopachrome and p-hydroxy-phenylpyruvic acid tautomerase activity (Lubetsky et al., 1999; Bendrat et al., 1997; Swope et al., 1998).

A correlation between tautomerase catalytic activity and MIF's cytokine activities is supported by several studies where the structure and tautomerization kinetics of homologues of human MIF from a parasitic nematode, Brugia malayi (Bm), were characterized. Bm MIF mutant P1-g is 10-fold less active in inducing production of TNF-α and chemotactic activity of human macrophages compared to the parent Bm MIF and human MIF (Zang et al., 2002). In addition, the P1-g mutant is greatly impaired in its ability to stimulate superoxide generation in activated neutrophils (Swope et al., 1998). Also a P1-a mutant of MIF, which loses the tautomerase activity, loses its ability to enhance matrix metal loproteinase (MMP) mRNA levels (Onodera et al., 2000). However, a mutation in the Pro-I region alone is not sufficient to abolish the glucocorticoid counter-regulation activity and monocyte chemotaxis inhibition (Bendrat et al., 1997; Hermanowski-Vosatka et al., 1999), and truncated MIF mutants also indicate a role for the carboxy terminus in MIF binding/activity (Kleemann et al., 2000; Mischke et al., 1997).

Activation of macrophages is an early step in inflammation and leads to an increase of pro-inflammatory cytokines, such as TNF, further resulting in tissue damage. MIF was initially, as the name suggests, shown to regulate macrophage migration. However, more recent studies have shown that a major activity of MIF is its ability to suppress the anti-inflammatory steroid response. Thus, the rationale for MIF as a therapeutic target is that blocking MIF attenuates the inflammatory cascade in sepsis and endotoxemia and improves the survival rate.

In several studies, administration of neutralizing anti-MIF antibody protects mice: (a) from LPS-induced lethality; (b) against lethal peritonitis and septic shock induced by E. coli peritonitis and (c) against lethal sepsis induced by cecal ligation (CLP) and puncture in TNF-α deficient mice.

The compound (S,R)-3-(4-hydroxyphenyl)-4,5-dihydro-5-isoxazole acetic acid methyl ester (ISO-1) was recently designed as an inhibitor of MIF activity (PCT Publication WO 02/100332). The crystal structure of MIF complexed to ISO-1 revealed that it binds to a hydrophobic pocket. In vitro, ISO-1 inhibits 60% of TNF release by LPS-treated macrophages. In vivo, intraperitoneal administration of ISO-1 at 40 mg/kg increased the survival rate in endotoxemia and sepsis (Al-Abed et al., 2005). These results are comparable with monoclonal anti-MIF antibodies for the treatment of septic animals.

The ISO-1 structure incorporates the structure of Schiff base inhibitors of MIF enzyme activity that were designed originally to mimic the structure of dopachrome tautomerization intermediates of MIF catalysis. While ISO-1 has moderate anti-inflammatory activity, synthesis of a focused library around the ISO-1 structure alone did not significantly improve MIF inhibitor activity. Thus, new molecular scaffolds are required to identify additional MIF inhibitors. The present invention addresses that need.

### SUMMARY OF THE INVENTION

Accordingly, the inventors have identified compounds that inhibit MIF. These compounds are useful for treating or preventing inflammation in mammals.

Thus, the invention is directed to compounds of formula I:

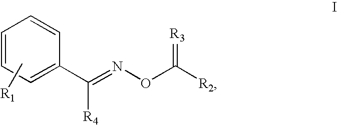

or a pharmaceutically acceptable salt, ester, or tautomer thereof,

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S; and

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

The invention is also directed to pharmaceutical compositions comprising above compounds, or a pharmaceutically acceptable salt thereof, in a pharmaceutically acceptable excipient.

The invention is additionally directed to a compound of formula I:

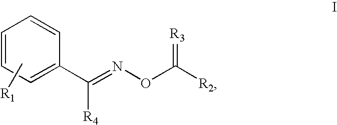

or a pharmaceutically acceptable salt, ester, or tautomer thereof,

wherein

R₁ is a single or multiple substitution independently H, OH, R₅, N(R₅), SR₅, or a halogen, wherein at least one substitution is a halogen;

R₂ comprises a ring structure in which an atom in the ring structure is bound to the carbon that is bound to R₃;

R₃ is O, C(R₅)₂, or S; and

R₄ is H, R₅, or a halogen, wherein R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. The invention also encompasses pharmaceutical compositions comprising any of these compounds.

Also, the invention is directed to a compound of formula I:

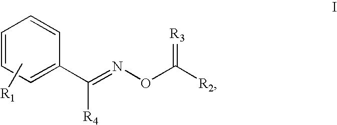

or a pharmaceutically acceptable salt, ester, or tautomer thereof,

where

R₁ is a single or multiple substitution independently H, OH, R₅, N(R₅), SR₅, or a halogen;

R₂ is para-hydroxymethylphenyl;

R₃ is O, C(R₅), or S; and

R₄ is H, R₅, or a halogen, wherein R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

The invention is further directed to a compound of formula III

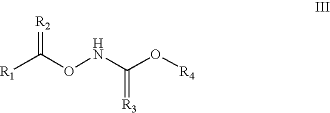

where

R₁ comprises a ring structure in which an atom in the ring structure is bound to the carbon that is bound to R₂;

R₂ and R3 are independently O, C(R₅)₂, or S;

R₄ is a straight or branched C₂-C₆ alkyl, a straight or branched C₂-C₆ alkenyl, a straight or branched C₂-C₆ alkanoyl, or a straight or branched C₂-C₆ alkoxy;

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. The invention also encompasses pharmaceutical compositions comprising any of these compounds.

Also, the invention is directed to methods of inhibiting macrophage migration inhibitory factor (MIF) activity in a mammal. The methods comprise administering any of the above-identified pharmaceutical compositions to the mammal in an amount effective to inhibit MIF activity in the mammal.

Additionally, the invention is directed to other methods of inhibiting macrophage migration inhibitory factor (MIF) activity in a mammal. The methods comprise administering a pharmaceutical composition to the mammal in an amount effective to inhibit MIF activity in the mammal. In these methods, the pharmaceutical composition comprises a compound of formula I or formula II, or a pharmaceutically acceptable salt, ester, or tautomer thereof, in a pharmaceutically acceptable excipient, where formula I and formula II are

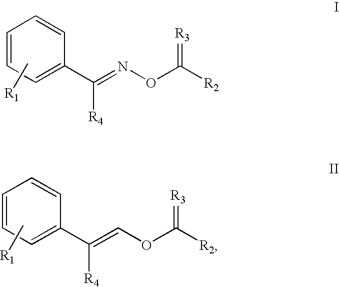

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S;

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C2-C6 alkoxy.

Further, the invention is directed to methods of treating or preventing inflammation in a mammal. The methods comprise administering any of the above-identified the pharmaceutical compositions to the mammal in an amount effective to treat or prevent the inflammation in the mammal.

The invention is also directed to other methods of treating or preventing inflammation in a mammal. The methods comprise administering a pharmaceutical composition to the mammal in an amount effective to treat or prevent the inflammation in the mammal, where the pharmaceutical composition comprises a compound of formula I or formula II, or a pharmaceutically acceptable salt, ester, or tautomer thereof, in a pharmaceutically acceptable excipient. Formula I and formula II are

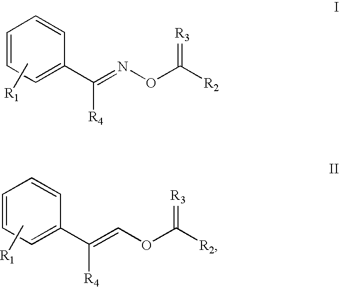

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S;

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

Additionally, the invention is directed to methods of treating a mammal having sepsis, septicemia, and/or endotoxic shock. The methods comprise administering any of the above-identified pharmaceutical compositions to the mammal in an amount sufficient to treat the sepsis, septicemia and/or endotoxic shock.

The invention is further directed to additional methods of treating a mammal having sepsis, septicemia, and/or endotoxic shock. The methods comprise administering a compound to the mammal in an amount sufficient to treat the sepsis, septicemia and/or endotoxic shock, where the compound is

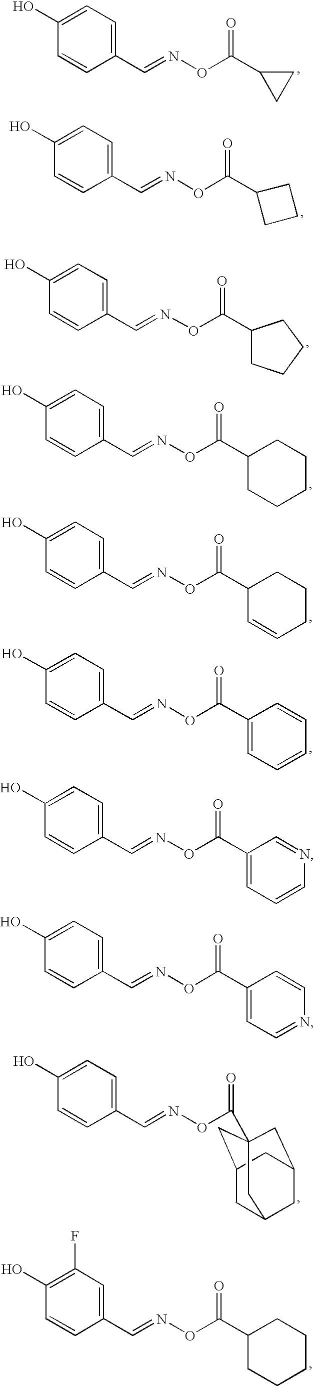

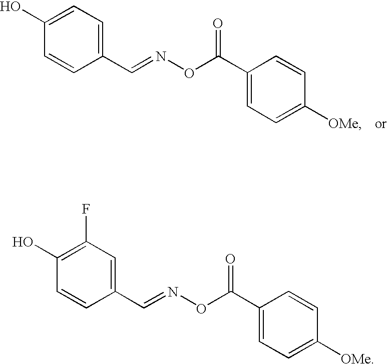

### BRIEF DESCRIPTION OF THE DRAWINGS

FIG. 1 shows the rationale for the synthesis of the Cyc-Oxi oxime compounds.

FIG. 2 shows three inhibitors of macrophage migration inhibitory factor (MIF).

FIG. 3 is a graph of experimental results establishing that Cyc-Oxi-11 suppresses the ability of MIF to regulate glucocorticoids in LPS-treated macrophages. Briefly, monocyte-derived macrophages from human peripheral blood were preincubated with dexamethasone (10⁻⁹) or dexamethasone plus MIF (3 nM purified native MIF) and various concentrations of Cyc-Oxi-11 (0, 0.01, 0.1 and 1 mM) before the addition of 0.5 μg/ml lipopolysaccharide (LPS). TNF-α production was then measured. The data shown are mean ±SD of triplicate wells in experiments that were repeated twice.

FIG. 4 is a graph of experimental results establishing that Cyc-Oxi-11 inhibits MIF induction of TNF release from LPS-stimulated macrophages. Briefly, monocyte-derived macrophages from human peripheral blood were pre-treated with various concentrations of Cyc-Oxi-11 10 minutes prior to the addition of 0.5 μg/ml (LPS). TNF-α production was then measured. The data shown are mean ±SD of triplicate wells in experiments that were repeated twice.

FIG. 5 is a graph of the kinetics of MIF appearance in the serum post CLP surgery.

FIG. 6 is a graph showing that Cyc-Oxi-II is protective even when given 24 h after the induction of sepsis.

FIG. 7 shows the synthesis and activity of compounds 3a-3h. IC₅₀ represents the inhibition of MIF tautomerase activity.

FIG. 8 shows the synthesis and activity of compounds 4-5. IC₅₀ represents the inhibition of MIF tautomerase activity.

### DETAILED DESCRIPTION OF THE INVENTION

As described in the Example, the inventors have identified compounds that inhibit MIF. These compounds are useful for treating or preventing inflammation in mammals.

Thus, the invention is directed to compounds of formula I:

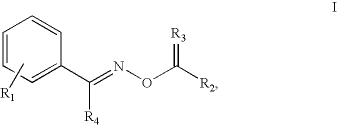

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S; and

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

Preferably, R₁ is H, OH or a halogen. More preferably, R₁ is OH. In the most preferred embodiments where R₁ a single substitution, R₁ is OH in the para position. Unless otherwise designated herein, the ortho, meta, or para designations of R₁ substituents of compounds of formula I designate the positions of the substituents in relation to the oxime (C═N—O—) moiety.

Where R₁ is multiple substitutions, one R₁ is preferably OH, most preferably in the para position, and the second substitution is preferably a halogen, more preferably fluorine. Most preferably this second substitution is a fluorine in the meta position.

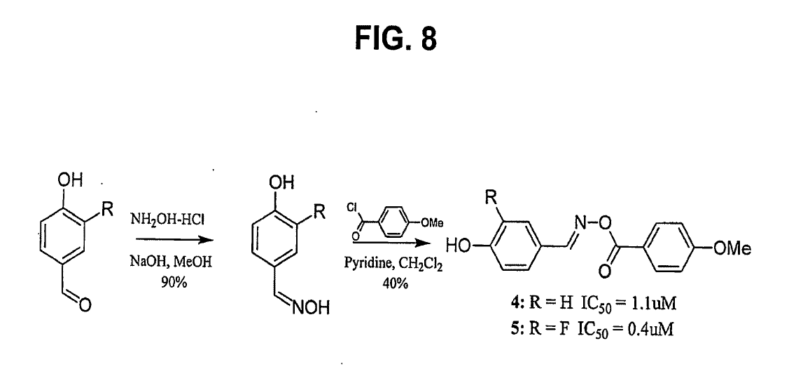

It is also preferred that R₂ comprises a 3-, 4-, 5- or 6-membered alicyclic, heterocyclic or aromatic ring. When R₂ is an alicyclic ring, the most preferred rings are cyclopropyl, cyclobutyl, 10 cyclopentyl, cyclohexyl, cyclohexyl-2,3-ene, or 1-adamantyl. When R₂ is a heterocyclic ring, the most preferred rings are pyrimidine, pyridazine, pyrazine, pyridine, pyrazole, imidazole, pyrrole, pyran or furan rings. When R₂ is an aromatic ring, the most preferred rings are cyclobenzyl, 4-pyrimidyl, 3-pyrimidyl, or para-hydroxymethylphenyl (the latter as in compounds 4 and 5 of FIG. 8).

The ring structure of R₂ can comprise more than one ring, and/or be substituted or unsubstituted. When the ring structure is substituted, it is preferably substituted with at least one straight or branched C₁-C₆ alkyl, straight or branched C₁-C₆ alkenyl, straight or branched C₁-C₆ alkanoyl, straight or branched C₁-C₆ alkoxy, keto, carboxy, nitro, amino, hydroxy, halogen, cyano, diazo, thio, or hydroxyamino. More preferred substitutions on R₂are at least one nitro, amino, hydroxyl or halogen.

Preferably, R₃ is O. It is also preferred that R₄ is H. In the most preferred compounds, R₃ is O and R₄ is H. Within those most preferred compounds, R₁ is preferably OH, most preferably in the para position. Also within the most preferred compounds where R₃ is O and R₄ is H, R₂ most preferably cyclopropyl, cyclobutyl, cyclopentyl, cyclohexyl, cyclohexyl-2,3-ene, cyclobenzyl, 4-pyrimidyl, 3-pyrimidyl, 1-adamantyl, or methoxyphenyl.

Specific preferred compounds comprise

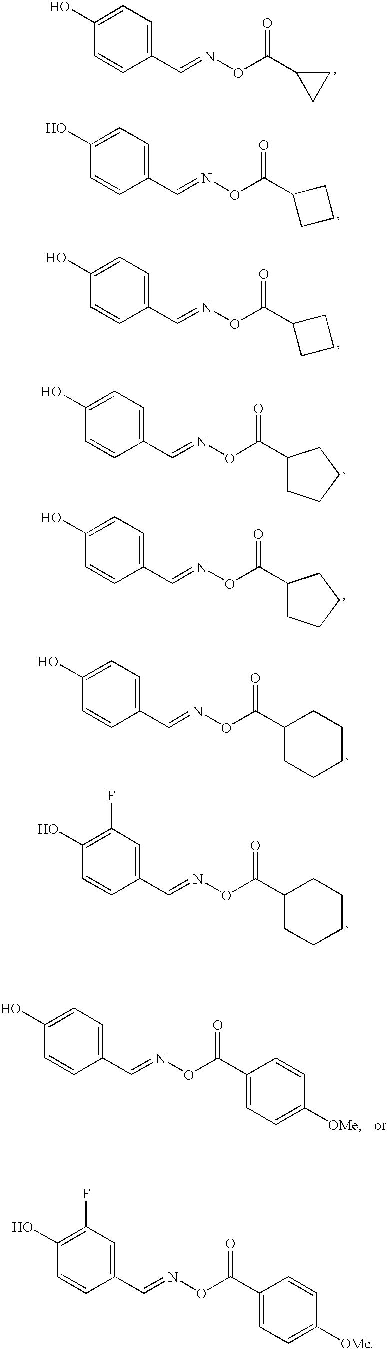

More preferably, the compound comprises, or consists of

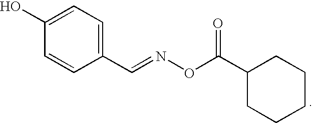

Other preferred compounds of the present invention comprise, or consist of

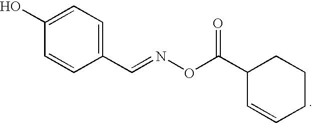

Still other preferred compounds of the present invention comprise, or consist of

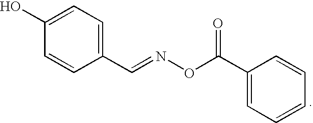

Additional preferred compounds comprise, or consist of

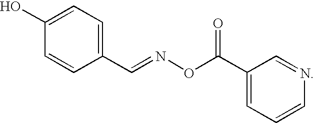

Further preferred compounds comprise, or consist of

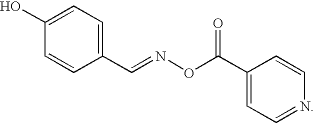

Still additional preferred compounds comprise, or consist of

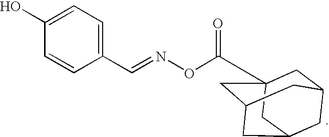

Further additional preferred compounds comprise, or consist of

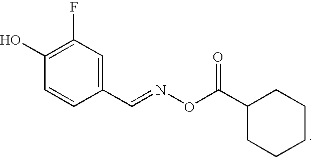

Still further additional preferred compounds comprise, or consist of

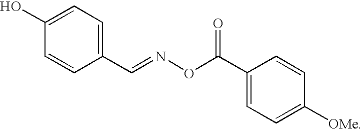

Additional preferred compounds comprise, or consist of

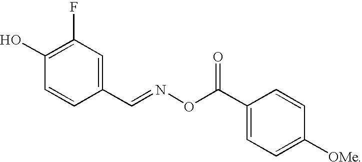

The above-described compounds are useful as inhibitors of macrophage migration inhibitory factor (MIF). Thus, the invention is also directed to pharmaceutical compositions comprising any of the above compounds, or a pharmaceutically acceptable salt, ester, or tautomer thereof, in a pharmaceutically acceptable excipient.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described compounds can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the compound. As used herein, nasally administering or nasal administration includes administering the compound to the mucous membranes of the nasal passage or nasal cavity of the patient. As used herein, pharmaceutical compositions for nasal administration of the compound include therapeutically effective amounts of the compound prepared by well-known methods to be administered, for example, as a nasal spray, nasal drop, suspension, gel, ointment, cream or powder. Administration of the compound may also take place using a nasal tampon or nasal sponge.

Where the compound is administered peripherally such that it must cross the blood-brain barrier, the compound is preferably formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The compound can be combined with one or several types of micelles, and can further be contained within the micelles or associated with their surface.

Alternatively, the compound can be combined with liposomes (lipid vesicles) to enhance absorption. The compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yioumas et al. Bile salts and their derivatives and detergent-like substances can also be included in the liposome formulation.

The invention is additionally directed to a compound of formula I:

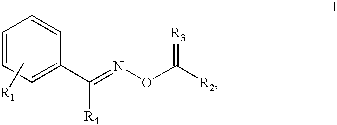

or a pharmaceutically acceptable salt, ester, or tautomer thereof,

wherein

R₁ is a single or multiple substitution independently H, OH, R₅, N(R₅), SR₅, or a halogen, wherein at least one substitution is a halogen;

R₂ comprises a ring structure in which an atom in the ring structure is bound to the carbon that is bound to R₃;

R₃ is O, C(R₅)₂, or S; and

R₄ is H, R₅, or a halogen, wherein

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. The invention also encompasses pharmaceutical compositions comprising any of these compounds.

Preferably, R₁ is a multiple substitution comprising OH and a halogen. More preferably R₁ comprises OH in the para position. It is also preferred that the halogen substitution is a fluorine. More preferably, the fluorine is in the meta position.

It is also preferred that R₂ comprises a 3-, 4-, 5- or 6-membered alicyclic, heterocyclic or aromatic ring. More preferably, the ring of R₂ is alicyclic. Most preferred alicyclic rings are cyclopropyl, cyclobutyl, cyclopentyl, cyclohexyl, cyclohexyl-2,3-ene, and 1-adamantyl.

Some of the preferred rings of R₂ are heterocyclic. A preferred heterocyclic ring is parahydroxymethylphenyl. Other preferred heterocyclic rings are pyrimidine, pyridazine, pyrazine, pyridine, pyrazole, imidazole, pyrrole, pyran and furan.

Others of the preferred rings of R₂ are aromatic. Most preferably the aromatic ring is cyclobenzyl, 4-pyrimidyl, or 3-pyrimidyl.

The ring structure of R₂ comprises more than one ring. Additionally, the ring structure of R₂ may be unsubstituted. Alternatively, the ring structure of R₂ is substituted with at least one straight or branched C₁-C₆ alkyl, straight or branched C₁-C₆ alkenyl, straight or branched C₁-C₆ alkanoyl, straight or branched C₁-C₆ alkoxy, keto, carboxy, nitro, amino, hydroxy, halogen, cyano, diazo, thio, or hydroxyamino. Other preferred substitutions of the ring structure of R₂ is at least one nitro, amino, hydroxyl or halogen.

Preferably with the invention compounds, R₃ is O. It is also preferred that R₄ is H. Most preferably R₃ is O and R₄ is H. With some preferred embodiments of these compounds, R₁ comprises an OH substitution and a halogen substitution. Preferably here, the OH is in the para position. More preferably here, R₂ is cyclopropyl, cyclobutyl, cyclopentyl, cyclohexyl, cyclohexyl-2,3-ene, cyclobenzyl, 4-pyrimidyl, 3-pyrimidyl, 1-adamantyl, or methoxyphenyl.

Preferably, the compound comprises, or consists of

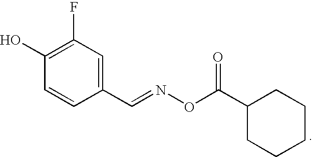

Other preferred compounds comprise, or consist of

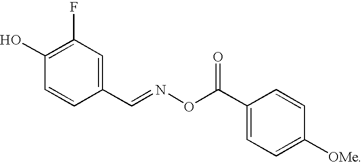

The invention is further directed to a compound of formula I:

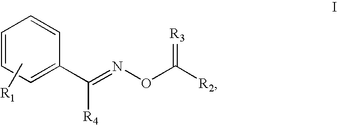

or a pharmaceutically acceptable salt, ester, or tautomer thereof,

wherein

R₁ is a single or multiple substitution independently H, OH, R₅, N(R₅), SR₅, or a halogen;

R₂ is para-hydroxymethylphenyl;

R₃ is O, C(R₅)₂, or S; and

R₄ is H, R₅, or a halogen, wherein

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

Preferably, R₁ is a multiple substitution comprising OH and a halogen. More preferably, OH in the para position and fluorine in the meta position. Most preferably, the compound comprises, or consists of

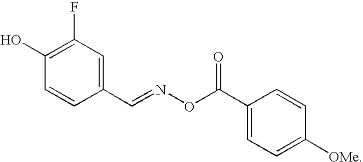

Another most preferred compound comprises, or consists of

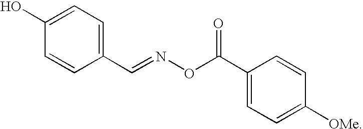

The invention is also directed to a pharmaceutical composition comprising any the above compounds, or a pharmaceutically acceptable salt, ester, or tautomer thereof, in a pharmaceutically acceptable excipient.

The invention is further directed to a compound of formula III

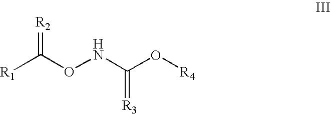

where

R₁ comprises a ring structure in which an atom in the ring structure is bound to the carbon that is bound to R₂;

R₂ and R₃ are independently O, C(R₅)₂, or S;

R₄ is a straight or branched C₂-C₆ alkyl, a straight or branched C₂-C₆ alkenyl, a straight or branched C₂-C₆ alkanoyl, or a straight or branched C₂-C₆ alkoxy;

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. The invention also encompasses pharmaceutical compositions comprising any of these compounds.

Preferably with these compounds, R₂ and R₃ are both O. Also preferably, R₄ is tert-butyl. Preferred R₁ moieties are cyclopropyl, cyclobutyl, cyclopentyl, cyclohexyl, cyclobenzyl, and a substituted cyclobenzyl. Where R₁ is a substituted cyclobenzyl, it is preferably a para-substituted cyclobenzyl. The para-substituted cyclobenzyl is most preferably

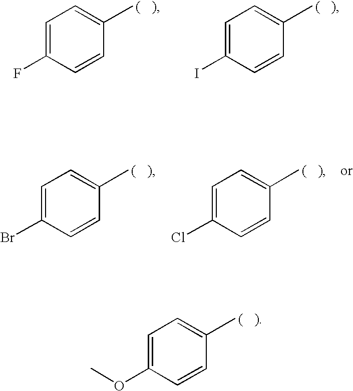

Even more preferably, the compound comprises

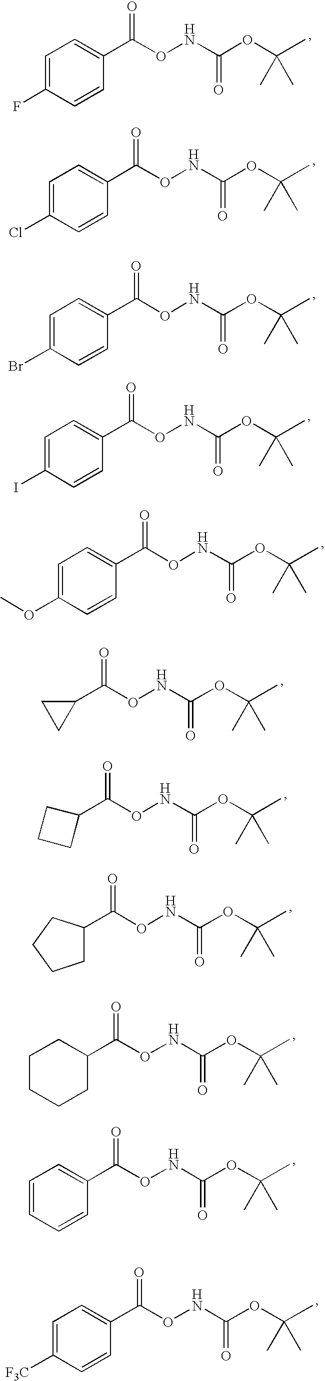

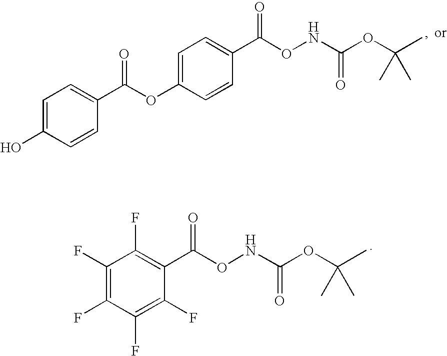

Still more preferably, the compound consists of

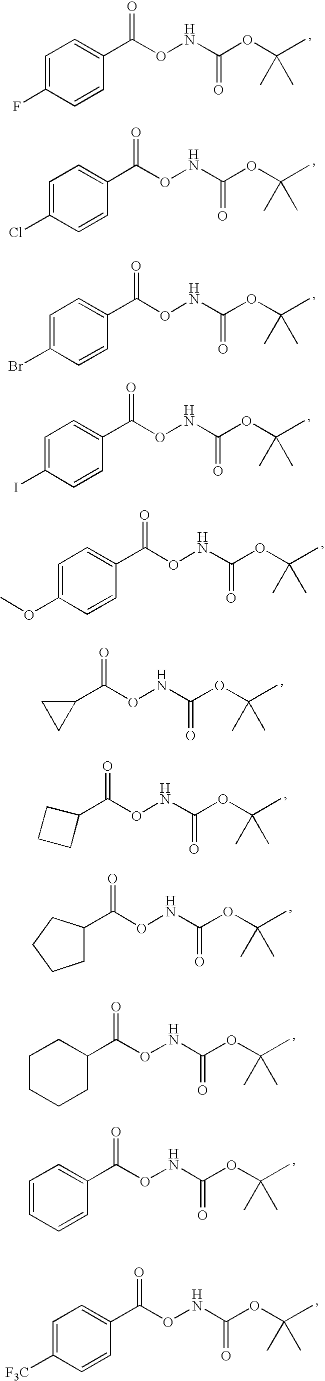

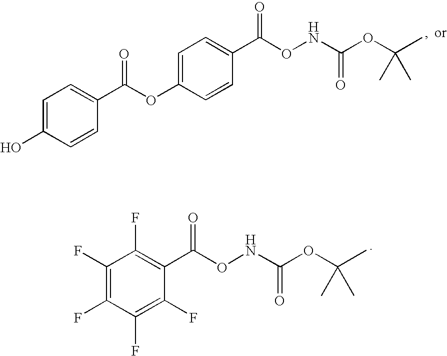

The compound most preferably comprises, or consists of

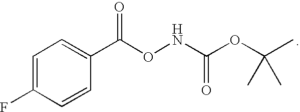

The compound can also most preferably comprise, or consist of

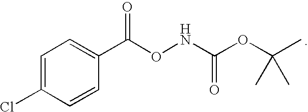

The compound can additionally most preferably comprise, or consist of

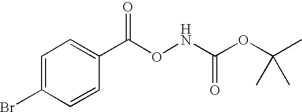

Additionally, the compound can most preferably comprise, or consist of

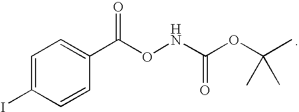

Further, the compound can most preferably comprise, or consist of

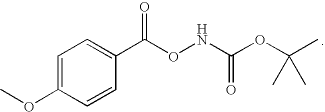

The compound can still further most preferably comprise, or consist of

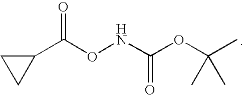

The compound can also most preferably comprise, or consist of

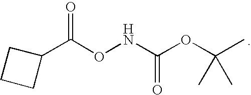

The compound can additionally preferably comprise, or consist of

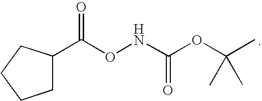

Additionally, the compound can most preferably comprise, or consist of

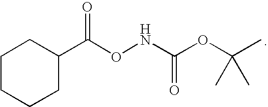

Further, the compound can most preferably comprise, or consist of

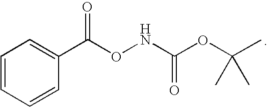

The invention also encompasses pharmaceutical composition comprising any of the above compounds, or a pharmaceutically acceptable salt, ester, or tautomer thereof, in a pharmaceutically acceptable excipient.

The invention is also directed to methods of inhibiting macrophage migration inhibitory factor (MIF) activity in a mammal. The methods comprise administering any of the above-identified pharmaceutical compositions to the mammal in an amount effective to inhibit MIF activity in the mammal.

Additionally, the invention is directed to other methods of inhibiting macrophage migration inhibitory factor (MIF) activity in a mammal. The methods comprise administering a pharmaceutical composition to the mammal in an amount effective to inhibit MIF activity in the mammal. In these methods, the pharmaceutical composition comprises a compound of formula I or formula II, or a pharmaceutically acceptable salt, ester, or tautomer thereof, in a pharmaceutically acceptable excipient, where formula I and formula II are

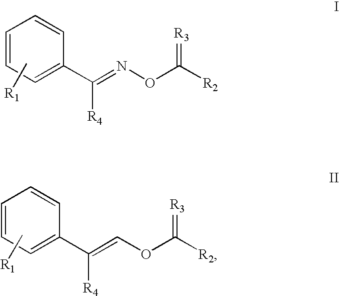

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S;

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. Preferably, the compound utilized in these methods is of formula I. It is also preferred if R₁ is OH in the para position. As with the compounds described above, R₂ preferably comprises a 3-, 4-, 5- or 6-membered alicyclic, heterocyclic or aromatic ring. More preferably, R₂ is cyclopropyl, cyclobutyl, cyclopentyl, cyclohexyl, cyclohexyl-2,3-ene, cyclobenzyl, 4-pyrimidyl, 3-pyrimidyl, 1-adamantyl, or methoxyphenyl.

Preferred specific compounds for the present methods include

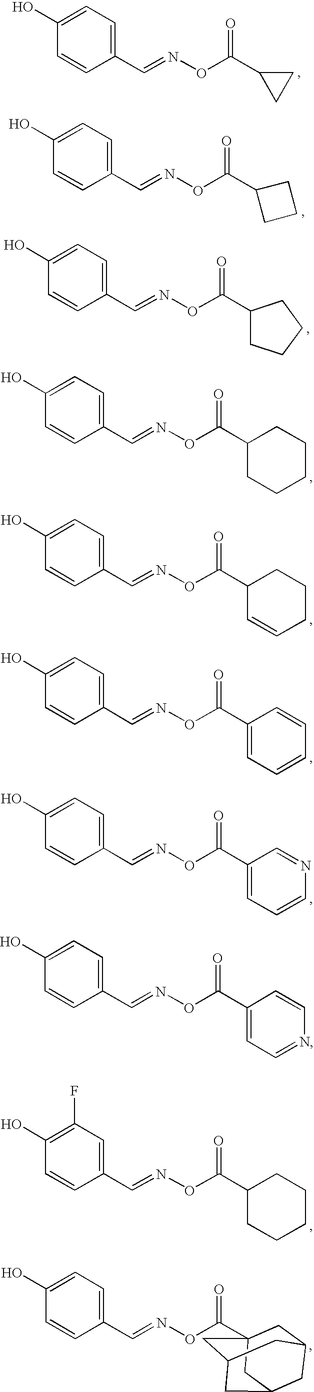

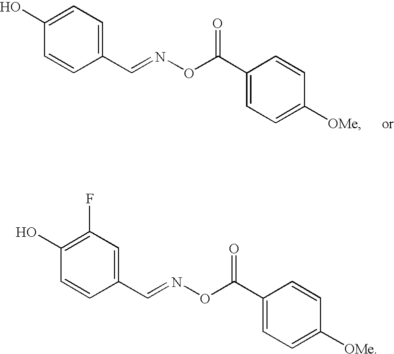

The mammal in these methods is preferably a human. The mammal also preferably has or is at risk for a condition that comprises an inflammatory cytokine cascade that is at least partially mediated by an MIF. Preferred examples of such conditions include cancer, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granulorna, granulomatosis, sarcoidosis, septic abortion, epididymitis, vaginitis, prostatitis, urethritis, bronchitis, emphysema, rhinitis, cystic fibrosis, pneumonitis, pneumoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, type 1 diabetes, type 2 diabetes, Berger's disease, Retier's syndrome, or Hodgkins disease. In the most preferred embodiments, the condition is sepsis, septicemia, and/or endotoxic shock.

The invention is also directed to methods of treating or preventing inflammation in a mammal. The methods comprise administering any of the above-identified pharmaceutical compositions to the mammal in an amount effective to treat or prevent the inflammation in the mammal.

The invention is additionally directed to other methods of treating or preventing inflammation in a mammal. The methods comprise administering a pharmaceutical composition to the mammal in an amount effective to treat or prevent the inflammation in the mammal, where the pharmaceutical composition comprises a compound of formula I or formula II, or a pharmaceutically acceptable salt, ester, or tautomer thereof, in a pharmaceutically acceptable excipient. Here, formula I and formula II are

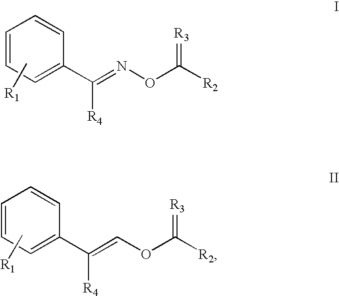

where

R₁ is a single or multiple substitution independently H, OH, R₅, N(R₅), SR₅, or a halogen;

R₂ comprises a ring structure in which an atom in the ring structure is bound to the carbon that is bound to R₃;

R₃ is O, C(R₅)₂, or S;

R₄ is H, R₅, or a halogen, where

R₅ is independently H, a straight or branched C₂-C₆ alkyl, a straight or branched C₂-C₆ alkenyl, a straight or branched C₂-C₆ alkanoyl, or a straight or branched C₂-C₆ alkoxy. As with the methods described above, the compounds in these methods are preferably of formula 1. It is also preferred if R₁ of the compounds is OH in the para position. Additionally, R₂ preferably comprises a 3-, 4-, 5- or 6-membered alicyclic, heterocyclic or aromatic ring. Most preferably, R₂ is cyclopropyl, cyclobutyl, cyclopentyl, cyclohexyl, cyclohexyl-2,3-ene, cyclobenzyl, 4-pyrimidyl, 3-pyrimidyl, 1-adamantyl, or methoxyphenyl. Preferred specific compounds for these methods are

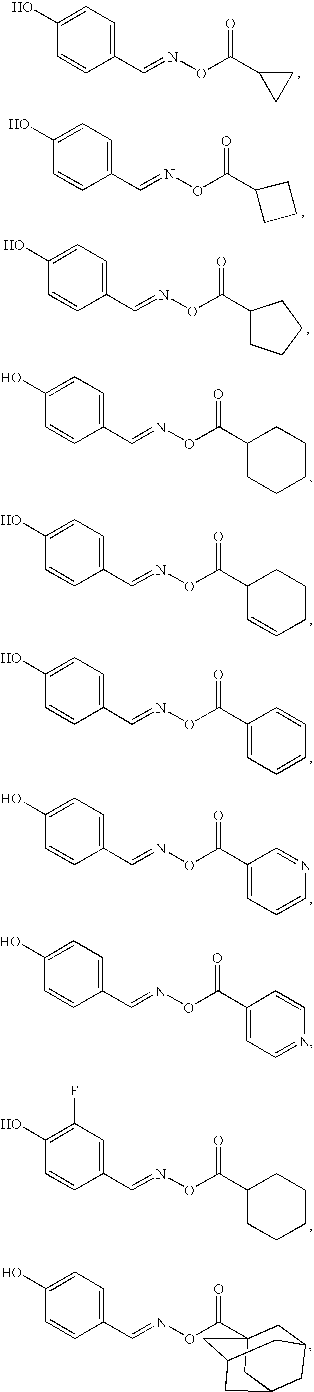

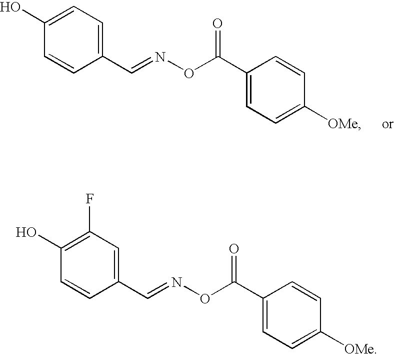

The mammal in these methods is preferably a human. The mammal also preferably has a condition that comprises an inflammatory cytokine cascade that is at least partially mediated by an MIF. Preferred examples of such conditions include cancer, acute respiratory distress syndrome, cytokine-mediated toxicity, psoriasis, interleukin-2 toxicity, appendicitis, peptic, gastric and duodenal ulcers, peritonitis,.pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Retier's syndrome, or Hodgkins disease. In the most preferred embodiments, the mammal has or is at risk for sepsis, septicemia, and/or endotoxic shock.

These methods can further comprise administering a second anti-inflammatory agent to the mammal. Nonlimiting examples of the second anti-inflammatory agent is an NSAID, a salicylate, a COX inhibitor, a COX-2 inhibitor, or a steroid. Preferably, the mammal has or is at risk for sepsis, septicemia, and/or endotoxic shock and the second treatment is administration of a muscarinic agonist, an adrenomedullin, an adrenomedullin binding protein, a milk fat globule epidermal growth factor factor VIII, an activated protein C, or an α_{2A}-adrenergic antagonist.

The invention is also directed to a method of treating a mammal having sepsis, septicemia, and/or endotoxic shock. The method comprises administering any of the above pharmaceutical compositions to the mammal in an amount sufficient to treat the sepsis, septicemia and/or endotoxic shock.

The invention is further directed to other methods of treating a mammal having sepsis, septicemia, and/or endotoxic shock. The methods comprise administering a compound to the mammal in an amount sufficient to treat the sepsis, septicemia and/or endotoxic shock, where the compound is

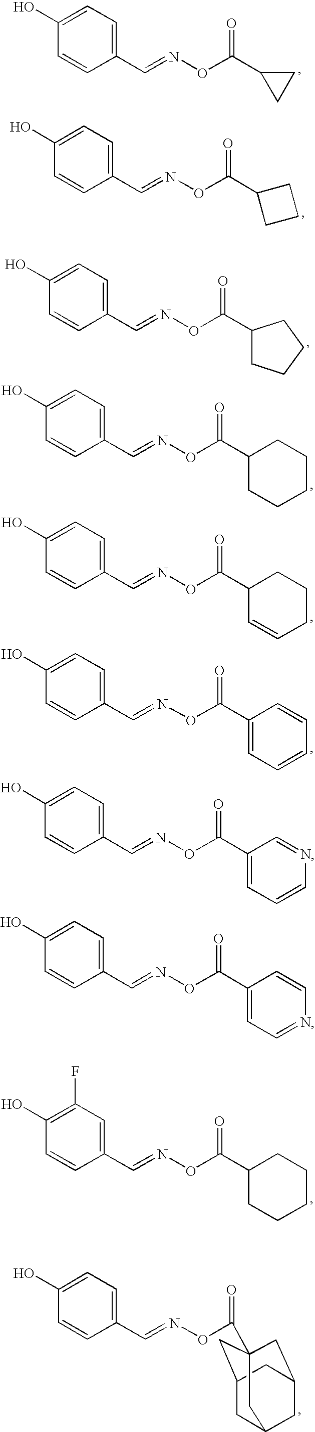

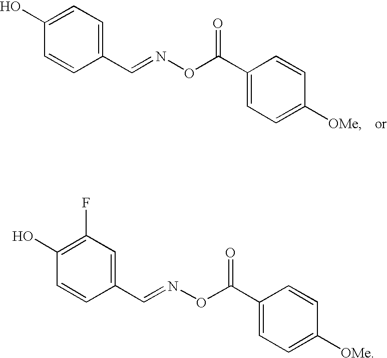

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### Oxime Inhibitors of Macrophage Migration Inhibitory Factor

One route for the design of inhibitors of MIF pro-inflammatory activity has focused on interfering with the MIF tautomerase active site to inhibit tautomerase activity. In this regard, disruption of the active site by insertion of an alanine between Pro-1 and Met-2 (pam) abolishes the tautomerase catalytic activity and the resultant mutant is defective in the in vitro glucocorticoid counter-regulatory activity of MIF (Lubetsky et al., 2002). Also, a P450-dependent metabolite of acetaminophen, N-acetyl-p-benzoquinone imine (NAPQI) covalently binds to MIF at its enzymatic site and inactivates MIF cytokine activity in a number of in vitro bioassays, including interference with the anti-inflammatory effect of dexamethasone, suggesting a role of the active site in mediating MIF bioactivity (Senter et al., 2002). Unfortunately, the toxicity of NAPQI prevents its use as a systemic MIF antagonist. Therefore, it was hypothesized that compounds mimicking the indole product of MIF's tautomerase catalysis could bind in the active site and be effective inhibitors. To achieve this goal, Schiff base adducts were synthesized by coupling amino acid methyl ester with p-hydroxybenzaldehyde to furnish an amino acid Schiff base-type compound, Type II. The most potent inhibitor was found to be tryptophan Schiff base with an IC₅₀ of 1.65 μM (Dios et al., 2002). Due to the slow rate of hydrolysis of tryptophan Schiff base in aqueous medium, additional phenylimine scaffolds were explored as potential MIF antagonists. Several representative phenylimine compounds were tested for dopachrome tautomerase inhibitory activity and it was concluded that the isoxazolines represent an attractive scaffold for further attention (Lubetsky et al., 2002). The lead inhibitor of MIF tautomerase and proinflammatory activity in this series is (S,R)-3-(4-hydroxyphenyl)-4,5-dihydro-5-isoxazole acetic acid methyl ester (ISO-1). The crystal structure of MIF complexed to ISO-1 revealed binding in the active site similar to p-hydroxy-phenylpyruvic acid. Further study of MIF bound with its inhibitor showed that active site occupation is associated with inhibition of MIF proinflammatory properties in vivo and in vitro, further establishing a role for the catalytic active site of MIF in inflammatory activities. These prior studies provided a molecular basis for the rational design of a new class of compounds resulting in the production of Cyc-Oxi-11, which in confirmatory testing is 30-fold more potent than ISO-1 in MIF tautomerase inhibition. In addition, Cyc-Oxi-11 lacks obvious toxicity at high doses, both in vitro and in vivo. Cyc-Oxi-11 and related compounds were evaluated as possible therapeutic agents for the treatment of sepsis.

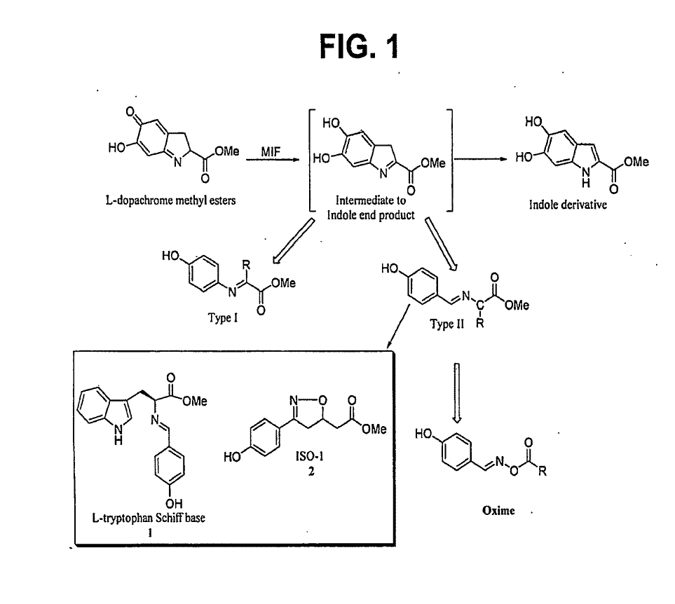

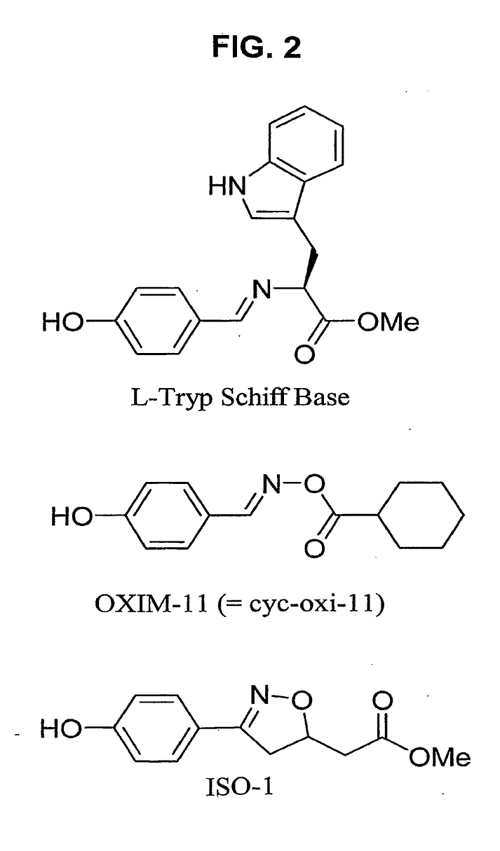

Design of Cyc-Oxi-11. ISO-1, the pharmacological inhibitor of MIF, neutralizes exogenous and endogenous MIF in in vitro and in vivo models. This indicates the successful application of chemical approaches to antagonize MIF biological activities. Although ISO-1 has significant anti-inflammatory activity, development of better inhibitors was desired. Synthesis of a focused library centered on the ISO-1 structure did not significantly improve the activity (data not shown). However, ISO-1 was integrated with two other scaffolds (FIG. 1) to design a new scaffold, an example of which is Cyc-Oxi-11 (=OXIM-11) (FIG. 2), which, along with its fluoridated derivative (Example 2) is a more potent inhibitor of MIF activity than any previously described compound. Cyc-Oxi-11 is one of 29 oxime derivatives that were synthesized around the scaffold and is 30-fold more potent inhibitor of MIF proinflammatory activity in vitro than ISO-1. Representative structures of the novel oxime scaffold are presented in Table I, with their IC₅₀ of inhibiting MIF dopachrome tautomerase activity. Since toxicity is a concern with respect to the proposed therapeutic use of any novel compound, preliminary acute toxicity screening of Cyc-Oxi-11 was conducted. No evidence of toxicity in intraperitoneal injection was found with doses up to 100 mg/Kg (data not shown). In preliminary studies, the antibacterial effect of Cyc-Oxi-11 was also tested and was found to be negative.

TABLE 1
Selected structures of Cyc-Oxi (OXIM) scaffold.
IC₅₀ represents the inhibition of MIF
tautomerase activity

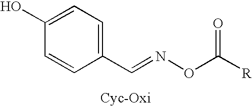

| R | IC₅₀(μM) | R | IC₅₀ |
|---|---|---|---|
| CH₃— | 85.0 | 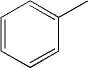 | 7 |
| 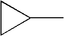 | 5.5 | 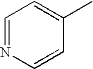 | 38 |
| 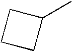 | 12.0 | 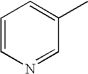 | 18 |
| 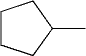 | 4.8 | | |
| 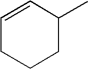 | 3.4 | | |
| 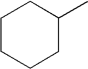 | 1.3 | | |
| 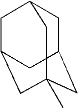 | 3.0 | | |

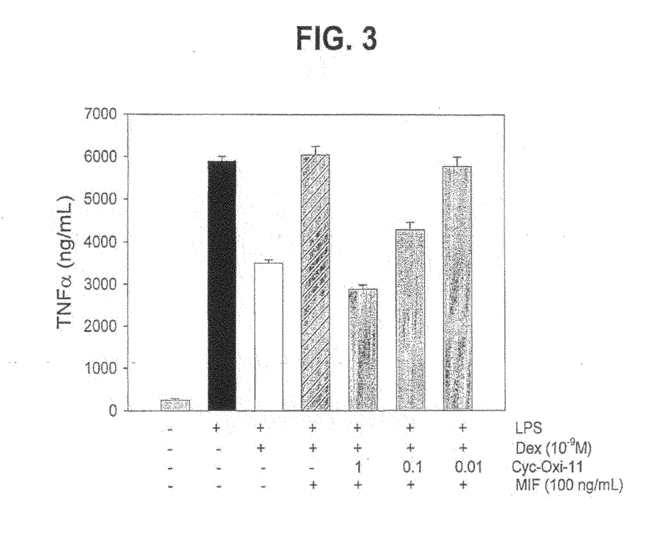

Cyc-Oxi-11 binding to the MIF active site down-regulates MIF glucocorticoid-regulating activity on LPS-activated monocytes. As shown with the study of ISO-1 above, the more potent neutralization of MIF proinflammatory activity in vitro is associated with enhanced inhibitory effect on MIF tautomerase activity. This association is further borne out in the new class of Cyc-Oxi agents. As shown in FIG. 3, Cyc-Oxi-11 significantly inhibited MIF-dependent interference with glucocorticoids from LPS-stimulated macrophages and Cyc-Oxi-11 is one of the most potent inhibitor of MIF tautomerase and proinflammatory activity with an IC₅₀ of ˜3 μM in both assays (30-fold more potent than ISO-1). Cyc-Oxi-11 inhibits TNF release in vitro.

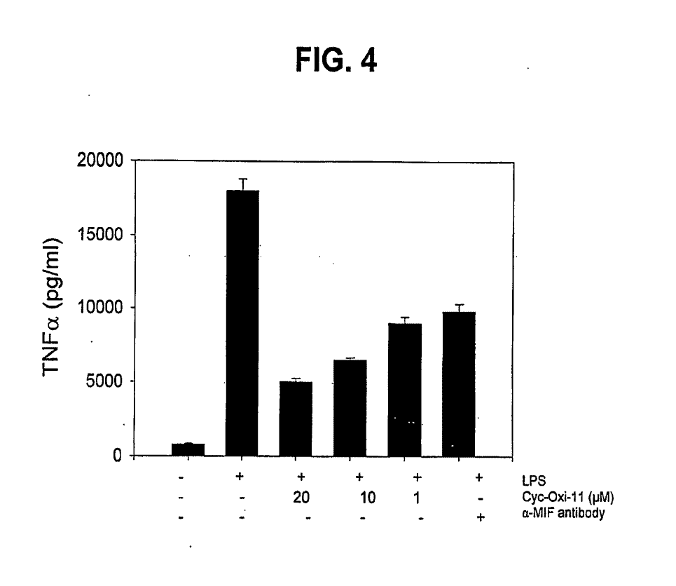

Cyc-Oxi-11 inhibits MIF proinflammatory activity in vitro. It has been shown that the macrophage is an abundant source of MIF (Calandra et al., 1994), which is released after LPS stimulation. This led to an examination of the autocrine and paracrine activity of secreted MIF in vitro. Previous studies showed that neutralization of MIF using antibodies blocked TNF secretion by LPS-stimulated macrophages. Here, it was determined whether Cyc-Oxi-11 neutralization of secreted MIF from LPS-stimulated human macrophages is able to inhibit MIF activity to mediate TNF release. As shown in FIG. 4, Cyc-Oxi-11, in a dose-dependent manner, inhibits TNF release by LPS-stimulated human macrophages similarly to anti-MIF antibody treatment.

Small molecules that bind at the catalytic site of MIF abrogate the inflammatory and glucocorticoid regulatory functions of the molecule. The rational design of inhibitors has resulted in the identification of Cyc-Oxi-11 and certain related OXIM scaffold compounds as the most potent inhibitor of MIF activity yet designed. Since the preliminary acute toxicity screening found no evidence of toxicity at doses up to 100 mg/kg this molecule shows potential as a therapeutic agent for reducing the devastating morbidity and mortality associated with sepsis, as well as other MIF-mediated diseases and conditions.

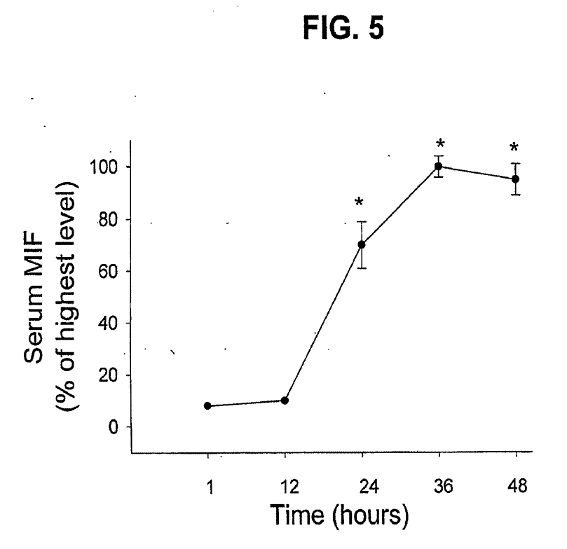

The activity of Cyc-Oxi-11 to prevent death by sepsis was next evaluated. The kinetics of MIF appearance in the serum post CLP surgery was determined by collecting blood after 6, 12, 24, 36 and 48 hours post CLP and then analyzing the serum by Western Blot to determine the circulating MIF levels. Five mice were tested pertime point. As shown in FIG. 5, serum MIF starts to increase after 12 hours and peaks at about 36 hours. *P<0.05.

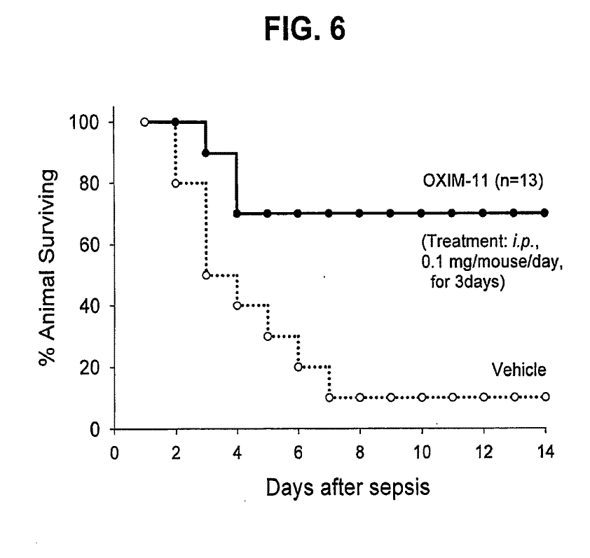

Other mice were then injected intraperitoneally with Cyc-Oxi-11 (0:1 mg/mouse/day) or vehicle 24 hours after CLP (n=13). An additional two injections were given, on day 2 and day 3. Thirteen mice were tested. The results are shown in FIG. 6. Cyc-Oxi-11 treatment 24 hours after onset of sepsis considerably reduced the deaths caused by CLP (P<0.001)

### Example 2

### Fluorination of OXIM-11 (Cyc-Oxi-11) Improves its Potent Inhibition of Macrophage Migration Inhibitory Factor Activity

### Example Summary

The synthesis of a series of halogenated (E)-4-hydroxybenzaldehyde O-cyclohexanecarbonyloxime (OXIM-11, FIG. 2) as potent and specific inhibitors of MIF tautomerase activity is described. Only mono-fluorination of the 4-hydroxy bearing phenyl ring of the OXIM scaffold improves the potency of the inhibitors, up to 63% compared to the parent compounds.

Macrophage migration inhibitory factor (MIF) is a pro-inflammatory cytokine that plays a critical role in the pathogenesis of inflammatory diseases. MIF, a homotrimer (Sun et al., 1996; Sugimoto et al., 1996), possesses the unique ability to catalyze the tautomerization of non-physiological substrates such as D or L-dopachrome methyl ester into their corresponding indole derivatives (Rosengren et al., 1996). Blocking this active site using either mutagenesis or small molecules inhibits MIF biological activity in sepsis (Beishuizen et al., 2001; Lue et al., 2002; Calandra et al., 2002; Calendra et al., 2000), EAN and type I diabetes (Cvetkovic et al., 2005).

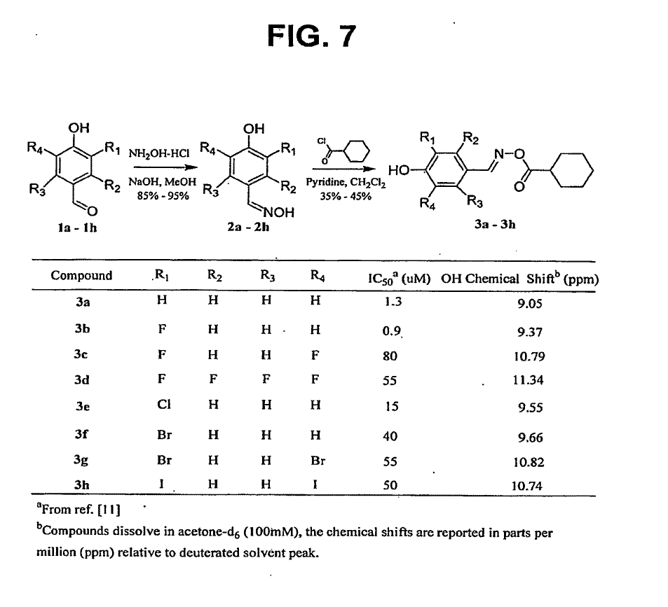

Recently, we further modified the target molecule to give the most potent inhibitor of MIF, (E)-4-hydroxybenzaldehyde Ocyclohexanecarbonyloxime (OXIM-11, cyc-oxi-11)(FIG. 2; compound 3a in FIG. 7). As established here, the molecule with cyclohexyl group (3a)(FIG. 7) and 4-Methoxyphenyl group (4, FIG. 8) (Scheme 2) have the most inhibition activity. 3a inhibits the dopachrome tautomerase activity with an IC₅₀ of 1.3 μM, and 4 inhibits the dopachrome tautomerase activity, with an IC₅₀ of 1.1 μM.

This Example describes the influence of ortho-halogenation in respect to the hydroxyl group on the potency to inhibit MIF tautomerase activity. Thus, the synthesis of a series of halogenated (E)-4-hydroxybenzaldehyde O-cyclohexanecarbonyloxime (OXIM-11, 3a), a potent and specific inhibitor of the MIF tautomerase activity is described. The mono-fluorination ortho to the hydroxyl improves the inhibition of MIF bioactivity up to 63%.

The halogenated 4-hydroxybenzaldehydes 1b-1h (FIG. 7) were either commercially available or prepared according to the procedure described in literature (Lawrence et al., 2003). The oximes 2a-2h (FIG. 7) were synthesized in excellent yields by condensation of hydroxylamine with the aldehydes 1a-1b in basic alcoholic solvent. The final compounds 3a-3h (FIG. 7) were synthesized in good yields by condensation of oximes 2a-2h with cyclohexanecarboxylic acid chloride in dry dichloromethane in present of pyridine from 0° C. to room temperature overnight (Scheme 1) (See Supplemental Material below). Compound 4 and 5 were prepared as the similar method as the compound 3 (FIG. 8). The final compounds 3a-3h, 4 and 5 reported here were fully characterized by ¹H NMR, ¹³C NMR and ESI-MS (See Supplemental Material below).

The crystal structure of MIF complexed to ISO-1 or OXIM-11 revealed that the phenolic group has a critical role in binding within the active site of MIF in both substrate and inhibitors. Compounds bearing halogens in an ortho position of the phenolic group were thus evaluated to determine whether the halogen enhances the hydrogen bond of the phenol for the additional binding within the active site of MIF. The candidate compounds 3a-3h were synthesized, and the inhibition of MIF dopachrome tautomerase activity is presented in FIG. 7.

The (E)-4-hydroxybenzaldehyde O-cyclohexanecarbonyloxime (OXIM-11, 3a), inhibits MIF dopachrome tautomerase activity with an IC₅₀ of 1.3 μM. Mono-fluorination on the ortho position of the phenolic group, compound 3b, improves the inhibition of the dopachrome tautomerase activity by 35%, to an IC₅₀ of 0.9 μM. Besides the steric effect, the strong electronegativity of the fluorine substituent polarizes the phenolic ring and enhances the OH hydrogen bond accepting ability that corresponds to the observed the most potency of compound 3b. Difluoro analogue 3c and tetrafluoro analogue 3d were considerably less potent than 3b because of the electrostatic repulsion of the fluorine groups (Malamas et al., 2004). For example, the 2,6-difluoro analogue 3c is most likely to have repulsion with the amide group of Asn-97. However, the other halogenated compounds bearing chlorine or bromine or iodine, compound 3e-3h, have reduced activity (FIG. 7). This finding is not surprising because the hydrogen bonds between the side-chain of Asn-97 and hydroxyl group are the key interaction within the MIF active site (Lubetsky et al., 1999). Introducing bulky halogens such as chlorine, bromine and iodine ortho to the hydroxyl group significantly alter the size of the molecule, and result in noticeably decreased ligand binding. Also, the intramolecular hydrogen bonds between OH and the halogens reduce the OH hydrogen bond donating ability as evidenced by increasing the acidity of OH in proton NMR analysis (FIG. 7) (Himo et al., 2000). Therefore, fluorine on the ortho position of the phenolic group on compound 3b has a critical and specific role for additional binding within the active site of MIF. The enhancement on the dopachrome tautomerase activity with mono-fluorination was also observed with (E)-3-fluoro-4-hydroxybenzaldehyde O-4′-methoxyphenyl carbonyloxime (5). That analog has a 63% improvement in the dopachrome tautomerase activity over the parent compound (4) (FIG. 8).

In conclusion, the mono-fluorination onto the ortho position to the hydroxyl group has a critical impact on ligand binding within the MIF active site.

### Supplementary Material

MIF tautomerase activity was measured by UV-Visible recording spectrophotometry (SHIMADZU, UV 1600U). A fresh stock solution of L-dopachrome methyl ester was prepared at 2.4 mM through oxidation of L3,4-dihydroxyphenylalanine methyl ester with sodium periodate. 1 μL of MIF solution (800-900 ng/mL) and 1 μL of a DMSO solution with various concentrations of the enzymatic inhibitor were added into a plastic cuvette (10 mm, 1.5 mL) containing 0.7 mL assay buffer (1× potassium phosphate, pH 7.2). The L-dopachrome methyl ester solution (0.3 mL) was added to the assay buffer mixture. Activity was determined at room temperature and the spectrometric measurements were made at λ=475 nm for 20 seconds by monitoring the rate of decolorization of L-dopachrome methyl ester in comparison to a standard solution.

General procedure for the synthesis of halogenated (E)-4-hydroxybenzaldehyde O-cyclobexanecarbonyloxime (3a-3h). A mixture of halogenated 4-hydroxybenzaldehyde oxime (2a-2h, 12.8 mmol) and cyclohexanecarboxylic acid chloride (13.4 mmol) in 70 mL dry dichloromethane was cooled to 0° C. To this suspension added pyridine (12.8 mmol) dropwise, which resulted in a pale yellow solution. The solution was stirred at 0° C. for 10 min and then was allowed to warm to room temperature for 18 hr. The mixture was diluted with CH₂Cl₂ and water and the layers were separated. The aqueous portion was washed with CH₂Cl₂, and the combined organic fraction was washed with saturated NaCl and dried over MgSO₄. Filtration and evaporation in vacuo afforded a white solid, which was purified by flash chromatography (40% EtOAc/hexanes). Crystallization from EtOAc/hexanes afforded the desired white solid product (3a-3h).

Analytical data for compounds 3a-3h. All solvents were HPLC-grade from Fisher Scientific. Silica gel (Selecto Scientific, 32-63 μm average particle size) was used for flash column chromatography (FCC). Aluminum-backed Silica Gel 60 with a 254 nm fluorescent indicator TLC plates were used. Spots on TLC plates were visualized under a short wavelength UV lamp or stained with 12 vapor. NMR spectra were preformed on a Jeol Eclipse 270 spectrometer at 270 MHz for ¹H NMR spectra and 67.5 MHz for ¹³C NMR spectra. Coupling constants are reported in Hertz (Hz), and chemical shifts are reported in parts per million (ppm) relative to the deuterated solvent peak. The coupling constants (J) are measured in Hertz (Hz) and assigned as s (singlet), d (doublet), t (triplet), m (multiplet) and br (broad). Low-resolution mass spectra were acquired using Thermofinnigan LCQ DecaXPplus quadrupole ion trap MS with negative-ion mode.

Analytical data of some selected compounds. Compound 3a: isolated as white solid product (38%). ¹H NMR (270 MHz, acetone-d6) δ 9.04 (br, 1H), 842 (s, 1H), 7.65 (d, J=8.7 Hz, 2H), 6.94 (d, J=8.7 Hz, 2H), 2.46 (m, 1H), 1.2-2.0 (m, 10H); ¹³C NMR (67.5 MHz, acetone-d6) δ 172.23, 160.56, 155.92, 130.06, 122.23, 115.88, 41.67, 25.19; ESI-MS m/z246 (M⁺).

Compound 3b: isolated as white solid product (40%). ¹H NMR (270 MHz, acetone-d6) δ 9.37 (br, 1H), 8.44 (s, 1H), 7.55 (m, 1H), 7.45 (m, 1H), 7.10 (m, 1H); 2.46 (m, 1H), 1.2-2.0 (m, 10H); ¹³C NMR (67.5 MHz, acetone-d6) δ 172.10, 155.16, 153.29, 149.72, 148.22, 125.75, 123.07, 118.18, 115.20, 41.61, 25.17; ESI-MS m/z 264 (M⁺). Compound 3c: isolated as white solid product (35%). ¹H NMR (270 MHz, acetone-d6) δ 10.79 (br, 1H), 8.16 (s, 1H), 7.40 (d, J=8.4 Hz, 2H), 2.76 (m, 1H), 1.2-2.0 (m, 10H); ESI-MS m/z 282 (M⁺). Compound 3d: isolated as white solid product (45%). ¹H NMR (270 MHz, acetone-d6) δ 11.34 (br, 1H), 8.22 (s, 1H), 2.85 (m, 1H), 1.2-2.0 (m, 10H); ¹³C NMR (67.5 MHz, acetone-d6) δ 171.50, 146.54, 142.89, 139.25, 138.02, 129.32, 110.44, 42.16, 24.84; ESI-MS m/z 321 (M⁺). Compound 3e: isolated as white solid product (40%). ¹H NMR (270 MHz, acetone-d6) δ 9.55 (br, 1H), 8.50 (s, 1H), 7.85 (d, J=2.0 Hz, 1H), 7.67 (dd, J₁=8.5 Hz, J₂=2.0 Hz, 1H), 7.18 (d, J=8.5 Hz, 1H), 2.55 (m, 1H), 1.2-2.0 (m, 10H); ESI-MS m/z 280 (M⁻). Compound 3f: isolated as white solid product (39%). ¹H NMR (270 MHz, acetone-d6) δ 9.66 (br, 1H), 844 (s, 1H), 7.94 (d, J=2.0 Hz, 1H), 7.66 (dd, J₁=8.4 Hz, J₂=2.0 Hz, 1H), 7.10 (d, J=8.4 Hz, 1H), 2.47 (m, 1H), 1.2-2.0 (m, 10H); ¹³C NMR (67.5 MHz, acetone-d6) δ 172.12, 156.93, 154.75, 133.09, 128.87, 124.01, 116.82, 110.04, 41.62, 25.18. Compound 3g: isolated as white solid product (37%). ¹H NMR (270 MHz, acetone-d₆) δ 10.82 (br, 1H), 8.15 (s, 1H), 7.93 (s, 2H), 2.73 (m, 1H), 1.2-2.0 (m, 10H). Compound 3h: isolated as white solid product (40%). ¹H NMR (270 MHz, acetone-d₆) δ 10.74 (br, 1H), 8.13 (s, 2H), 8.10 (s, 1H), 2.71 (m, 1H), 1.2-2.0 (m, 10H). Compound 4: isolated as white solid product (40%). ¹H NMR (300 MHz, acetone-d₆) δ 9.05 (br, 1H), 8.60 (s, 1H), 8.02 (d, J=8.7 Hz, 2H), 7.68 (d, J=8.7 Hz, 2H), 7.05 (d, J=8.7 Hz, 2H), 6.93 (d, J=8.7 Hz, 2H), 3.87 (s, 3H); ESI-MS m/z 270 (M⁻). Compound 5: isolated as white solid product (40%). ¹H NMR (300 MHz, acetoned6) δ 9.37 (br, 1H), 8.62 (s, 1H), 8.02 (d, J=8.4 Hz, 2H), 7.60 (dd, J₁=8.4 Hz, J₂=2.0 Hz, 1H), 7.47 (d, J=8.4 Hz, 1H), 7.06 (m, 3H), 3.87 (s, 3H); ESI-MS m/z 288 (M⁻).

### Example 3

### Additional Compounds Inhibiting MIF

Additional compounds were produced and tested for MIF inhibitory activity in vitro by the methods described in the above examples. Table 2 provides the results of those experiments.

TABLE 2
Additional MIF inhibitors.

| Compound | Structure | IC₅₀ (μM) |
|---|---|---|
| BKII-91 | 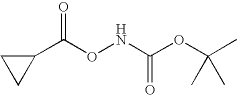 | 1.9 |
| BKII-92 | 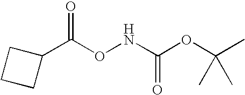 | 0.37 |
| BKII-93 | 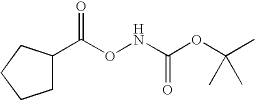 | 0.7 |
| BKII-85 | 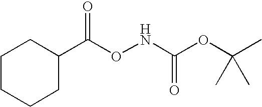 | 19.5 |
| BKII-90 | 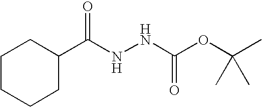 | 60 |
| BKII-98 | 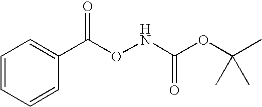 | 0.23 |
| BKII-99 | 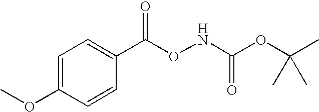 | 1.2 |
| BKIII-9W | 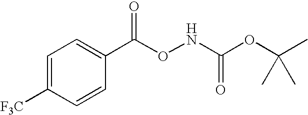 | 69 |
| BKIII-6Y | 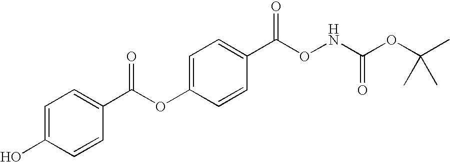 | 50 |
| BKIII-8W | 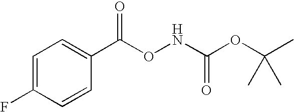 | 0.27 |
| BKIII-8Y | 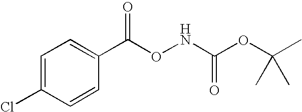 | 0.87 |
| BKIII-11Y | 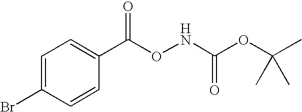 | 1.30 |
| BKIII-9Y | 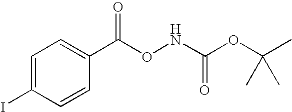 | 1.7 |
| BKIII-6W | 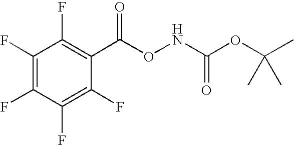 | 118 |

### REFERENCES

Abraham E: Why immunomodulatory therapies have not worked in sepsis. Intensive Care Med 25:556-566, 1999

Angus D C, Linde-Zwirble W T, Lidicker J, Clermont G, Carcillo J, Pinsky M R: Epidemiology of severe sepsis in the United States: analysis of incidence, outcome, and associated costs of care. Crit Care Med 29:1303-1310, 2001

Al-Abed Y, Dabideen D, Aijabari B, Valster A, Messmer D, Ochani M, Tanovic M, Ochani K, Bacher M, Nicoletti F, Metz C, Pavlov V A, Miller E J, Tracey K T: ISO-1 Binding to the Tautomerase Active Site of MIF Inhibits Its Pro-inflammatory Activity and Increases Survival in Severe Sepsis. J. Biol. Chem.280: 36541-36544, 2005.

Al-Abed Y, Lajabari B, Cheng K, Miller E: Inhibition of macrophage migration inhibitory factor (MIF) by Cyc-Oxi-11 increases survival in sepsis. Abstract for 28^{th} Annual Conference on Shock, Jun. 4-7, 2005, Marco Island, Fla.

Bacher M, Metz C N, Calandra T, Mayer K, Chesney J, Lohoff M, Gemsa D, Donnelly T, Bucala R: An essential regulatory role for macrophage migration inhibitory factor in T-cell activation. Proc Natl Acad Sci USA 93:7849-7854, 1996

Beishuizen A, Thijs L G, Haanen C, Vermes I: Macrophage migration inhibitory factor and hypothalamo-pituitary-adrenal function during critical illness. J Clin Endocrinol Metab 86:2811-2816, 2001

Bendrat K, Al-Abed Y, Callaway D J, Peng T, Calandra T, Metz C N, Bucala R: Biochemical and mutational investigations of the enzymatic activity of macrophage migration inhibitory factor. Biochemistry 36:15356-15362, 1997

Bernhagen J, Calandra T, Mitchell R A, Martin S B, Tracey K J, Voelter W, Manogue K R, Cerami A, Bucala R: MIF is a pituitary-derived cytokine that potentiates lethal endotoxaemia. Nature 365:756-759, 1993

Bernhagen J, Mitchell R A, Calandra T, Voelter W, Cerami A, Bucala R: Purification, bioactivity, and secondary structure analysis of mouse and human macrophage migration inhibitory factor (MIF). Biochemistry 33:14144-14155, 1994

Bloom B R, Bennett B:Mechanism of a reaction in vitro associated with delayed-type hypersensitivity. Science 153:80-82, 1966

Bozza M, Satoskar A R, Lin G, Lu B, Humbles A A, Gerard C, David J R: Targeted disruption of migration inhibitory factor gene reveals its critical role in sepsis. J Exp Med 189:341-346, 1999

Calandra T: Pathogenesis of septic shock: implications for prevention and treatment. J Chemother 13 Spec No 1:173-180, 2001

Calandra T, Bucala R: Macrophage migration inhibitory factor (MIF): a glucocorticoid counter-regulator within the immune system. Crit Rev Immunol 17:77-88, 1997

Calandra T, Bemhagen J, Mitchell R A, Bucala R: The macrophage is an important and previously unrecognized source of macrophage migration inhibitory factor. J Exp Med 179:1895-1902, 1994

Calandra T, Bemhagen J, Metz C N, Spiegel L A, Bacher M, Donnelly T, Cerami A, Bucala R: MIF as a glucocorticoid-induced modulator of cytokine production. Nature 377:68-71, 1995

Calandra T, Spiegel L A, Metz C N, Bucala R: Macrophage migration inhibitory factor is a critical mediator of the activation of immune cells by exotoxins of Gram-positive bacteria. Proc Natl Acad Sci USA 95:11383-11388, 1998

Calandra T, Echtenacher B, Roy D L, Pugin J, Metz C N, Hultner L, Heumann D, Mannel D, Bucala R, Glauser M P: Protection from septic shock by neutralization of macrophage migration inhibitory factor. Nat Med 6:164-170, 2000

Cvetkovic I, Al-Abed Y, Miljkovic D, Maksimovic-Ivanic D, Roth J, Bacher M, Lan H Y, Nicoletti F, Stosic-Grujicic S. Endocrinology 146:2942, 2005

David J: Delayed hypersensitivity in vitro: its mediation by cell-free substances formed by lymphoid cell-antigen interaction. Proc Natl Acad Sci USA 56:72-77, 1966

Dios A, Mitchell R A, Aljabari B, Lubetsky J, O'Connor K, Liao H, Senter P D, Manogue K R, Lolis E, Metz C, Bucala R, Callaway D J, Al-Abed Y: Inhibition of MIF bioactivity by rational design of pharmacological inhibitors of MIF tautomerase activity. J Med Chem 45:2410-2416, 2002

Donnelly S C, Haslett C, Reid P T, Grant I S, Wallace W A, Metz C N, Bruce L J, Bucala R: Regulatory role for macrophage migration inhibitory factor in acute respiratory distress syndrome. Nat Med 3:320-323, 1997

Echtenacher B, Falk W, Mannel D N, Krammer P H: Requirement of endogenous tumor necrosis factor/cachectin for recovery from experimental peritonitis. J Immunol 145:3762-3766, 1990

Friedman G, Silva E, Vincent J L: Has the mortality of septic shock changed with time. Crit Care Med 26:2078-2086, 1998

George M, Vaughan J H: In vitro cell migration as a model for delayed hypersensitivity. Proc Soc Exp Biol Med 111:514-521, 1962

Hayashi, S., Kurdowska, A, Stevens, M. D., Cohen, A. B., Stevens, M. D., Fujisawa, N. Miller, E. J. A synthetic inhibitor for α-chemokines inhibits the growth of melanoma cell lines. J. Clin. Invest.99: 2581-2587, 1997

Hermanowski-Vosatka A, Mundt S S, Ayala J M, Goyal S, Han Ion W A, Czerwinski R M, Wright S D, Whitman C P: Enymatically inactive macrophage migration inhibitory factor inhibits monocyte chemotaxis and random migration. Biochemistry 38:12841-12849, 1999

Himo F, Eriksson L A, Blomberg M R A, Siegbahn P E M. International Journal of Quantum Chemistry 76:714, 2000

Kato Y, Muto T, Tomura T, Tsumura H, Watarai H, Mikayama T, Ishizaka K, Kuroki R: The crystal structure of human glycosylation-inhibiting factor is a trimeric barrel with three 6-stranded beta-sheets. Proc Natl Acad Sci USA 93:3007-3010, 1996

Kleemann R, Rorsman H, Rosengren E, Mischke R, Mai N T, Bemhagen J: Dissection of the enzymatic and immunologic functions of macrophage migration inhibitory factor—Full immunologic activity of N-terminally truncated mutants. European Journal of Biochemistry 267:7183-7192, 2000a

Kleemann R, Hausser A, Geiger G, Mischke R, Burger-Kentischer A, Flieger O, Johannes F J, Roger T, Calandra T, Kapumiotu A, Grell M, Finkelmeler D, Brunner H, Bernhagen J: Intracellular action of the cytokine MIF to modulate AP-1 activity and the cell cycle through Jab1. Nature 408:211-216, 2000b

Lan H Y, Bacher M, Yang N, Mu W, Nikolic-Paterson D J, Metz C, Meinhardt A, Bucala R, Atkins R C: The pathogenic role of macrophage migration inhibitory factor in immunologically induced kidney disease in the rat. J Exp Med 185:1455-1465, 1997

Lawrence N J, Hepword L A, Rennison D, McGown A T, Hadfield J A J. Fluor. Chem. 123:101, 2003.

Leech M, Metz C, Santos L, Peng T, Holdsworth S R, Bucala R, Morand E F: Involvement of macrophage migration inhibitory factor in the evolution of rat adjuvant arthritis. Arthritis Rheum 41:910-917, 1998

Leng L, Metz C N, Fang Y, Xu J, Donnelly S, Baugh J, Delohery T, Chen Y, Mitchell R A, Bucala R: MIF signal transduction initiated by binding to CD74. J Exp Med 197:1467-1476, 2003

Lolis E, Bucala R: Crystal structure of macrophage migration inhibitory factor (MIF), a glucocorticoid-induced regulator of cytokine production, reveals a unique architecture. Proc Assoc Am Physicians 108:415-419, 1996

Lubetsky J B, Swope M, Dealwis C, Blake P, Lolis E: Pro-1 of macrophage migration inhibitory factor functions as a catalytic base in the phenylpyruvate tautomerase activity. Biochemistry 38:7346-7354, 1999

Lubetsky J B, Dios A, Han J, Aljabari B, Ruzsicska B, Mitchell R, Lolis E, Al-Abed Y: The tautomerase active site of macrophage migration inhibitory factor is a potential target for discovery of novel anti-inflammatory agents. J Biol Chem 277:24976-24982, 2002

Lue H, Kleemann R, Calandra T, Roger T, Bemhagen J: Macrophage migration inhibitory factor (MIF): mechanisms of action and role in disease. Microbes Infect 4:449-460, 2002

Malamas M S, Manas E S, McDevitt R E, Gunawan I, Xu Z B, Collini M D, Miller C P, Dinh T, Hunderson R A, Keith Jr. J C, Harris H A, J. Med. Chem. 47:5021, 2004.

Martin G S, Mannino D M, Eaton S, Moss M: The epidemiology of sepsis in the United States from 1979 through 2000. N Engl J Med 348:1546-1554, 2003.

Matsunaga J, Sinha D, Pannell L, Santis C, Solano F, Wistow G J, Hearing V J: Enzyme activity of macrophage migration inhibitory factor toward oxidized catecholamines. Journal of Biological Chemistry 274:3268-3271, 1999a

Matsunaga I, Sinha D, Solano F, Santis C, Wistow G, Hearing V: Macrophage migration inhibitory factor (MIF)—Its role in catecholamine metabolism. Cellular and Molecular Biology 45:1035-1040, 1999b

Mikulowska A, Metz C N, Bucala R, Holmdahl R: Macrophage migration inhibitory factor is involved in the pathogenesis of collagen-type II-induced arthritis in mice. J Immunol 158:5514-5517, 1997

Miller E. J., L. Ulloa, Lin X, Mantell L., Hu M., Yang H., L. J., Liao H., Romashko III J., Franek W., Tracey, K. J., and Simms. H. H. 2003. □-Chemokine receptor inhibitor reduces HMGB-1 induced acute lung injury. Shock 19:270.

Mischke R, Gessner A, Kapurniotu A, Juttner S, Kleemann R, Brunner H, Bernhagen J: Structure activity studies of the cytokine macrophage migration inhibitory factor (MIF) reveal a critical role for its carboxy terminus. FEBS Lett 414:226-232, 1997

Mitchell R A, Liao H, Chesney J, Fingerle-Rowson G, Baugh J, David J, Bucala R: Macrophage migration inhibitory factor (MIF) sustains macrophage proinflammatory function by inhibiting p53: regulatory role in the innate immune response. Proc Natl Acad Sci USA 99:345-350, 2002

O'Brien J M Jr, Abraham, E. New approaches to the treatment of sepsis. Clin Chest Med 24:521-48, 2003.

Onodera S, Kaneda K, Mizue Y, Koyama Y, Fujinaga M, Nishihira J: Macrophage migration inhibitory factor up-regulates expression of matrix metalloproteinases in synovial fibroblasts of rheumatoid arthritis. J Biol Chem 275:444-450, 2000

Orita M, Yamamoto S, Katayama N, Aoki M, Takayama K, Yamagiwa Y, Seki N, Suzuki H, Kurihara H, Sakashita H, Takeuchi M, Fujita S, Yamada T, Tanaka A: Coumarin and chromen-4-one analogues as tautomerase inhibitors of macrophage migration inhibitory factor: Discovery and X-ray crystallography. Journal of Medicinal Chemisty 44:540-547, 2001

Remick D G, Newcomb D E, Bolgos O L, Call D R: Comparison of the mortality and inflammatory response of two models of sepsis: lipopolysaccharide vs. cecal ligation and puncture. Shock 13:110-116, 2000

Roger T, David J, Glauser M P, Calandra T: MIF regulates innate immune responses through modulation of Toll-like receptor 4. Nature 414:920-924, 2001

Rosengren E, Bucala R, Aman P, Jacobsson L, Odh G, Metz C N, Rorsman H: The immunoregulatory mediator macrophage migration inhibitory factor (MIF) catalyzes a tautomerization reaction. Mol Med 2:143-149, 1996

Rosengren E, Aman P, Thelin S, Hansson C, Ahifors S, Bjork P, Jacobsson L, Rorsman H: The macrophage migration inhibitory factor MIF is a phenylpyruvate tautomerase. FEBS Lett 417:85-88, 1997

Sampey A V, Hall P H, Mitchell R A, Metz C N, Morand E F: Regulation of synoviocyte phospholipase A2 and cyclooxygenase 2 by macrophage migration inhibitory factor. Arthritis Rheum 44:1273-1280, 2001

Senter P D, Al-Abed Y, Metz C N, Benigni F, Mitchell R A, Chesney J, Han J, Gartner C G, Nelson S D, Todaro G J, Bucala R: Inhibition of macrophage migration inhibitory factor (MIF) tautomerase and biological activities by acetaminophen metabolites. Proc Natl Acad Sci USA 99:144-149, 2002

Sugimoto H, Suzuki M, Nakagawa A, Tanaka I, Fujinaga M, Nishihira J: Crystallization of rat liver macrophage migration inhibitory factor for MAD analysis. J Struct Biol 115:331-334, 1995

Sugimoto H, Suzuki M, Nakagawa A, Tanaka I, Nishihira J: Crystal structure of macrophage migration inhibitory factor from human lymphocyte at 2.1 A resolution. FEBS Lett 389:145-148, 1996

Sun H W, Bernhagen J, Bucala R, Lolis E: Crystal structure at 2.6-A resolution of human macrophage migration inhibitory factor. Proc Natl Acad Sci USA 93:5191-5196, 1996

Suzuki M, Murata E, Tanaka I, Nishihira J, Sakai M: Crystallization and a preliminary X-ray diffraction study of macrophage migration inhibitory factor from human lymphocytes. J Mol Biol 235:1141-1143, 1994

Suzuki M, Sugimoto H, Nakagawa A, Tanaka I, Nishihira J, Sakai M: Crystal structure of the macrophage migration inhibitory factor from rat liver. Nat Struct Biol 3:259-266, 1996

Swope M, Sun H W, Blake P R, Lolis E: Direct link between cytokine activity and a catalytic site for macrophage migration inhibitory factor. EMBO J 17:3534-3541, 1998

Taylor A B, Johnson W H, Czerwinski R M, Li H S, Hackert M L, Whitman C P: Crystal structure of macrophage migration inhibitory factor-complexed with (E)-2-fluoro-p-hydroxycinnamate at 1.8 Å resolution: Implications for enzymatic catalysis and inhibition. Biochemisiy 38:7444-7452, 1999

Wang H, Bloom O, Zhang M, Vishnubhakat J M, Ombrellino M, Che J, Frazier A, Yang H, Ivanova S, Borovikova L, Manogue K R, Faist E, Abraham E, Andersson J, Andersson U, Molina P E, Abumrad N N, Sama A, Tracey K J: HMG-1 as a late mediator of endotoxin lethality in mice. Science 285:248-251, 1999

Zang X, Taylor P, Wang J M, Meyer D J, Scott A L, Walkinshaw M D, Maizels R M: Homologues of human macrophage migration inhibitory factor from a parasitic nematode: Gene cloning, protein activity and crystal structure. J. Biol. Chem.277:44261-44267, 2002

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A compound of formula I: 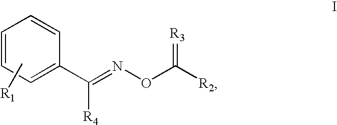
or a pharmaceutically acceptable salt, ester, or tautomer thereof,
wherein
R₁ is a single or multiple substitution independently OH or a halogen;
R₂ comprises a ring structure in which an atom in the ring structure is bound to the carbon that is bound to R₃;
R₃ is O, C(R₅)₂, or S; and
R₄ is H, R₅, or a halogen, wherein
R₅ is independently H, a straight or branched C₂-C₆ alkyl, a straight or branched C₂-C₆ alkenyl, a straight or branched C₂-C₆ alkanoyl, or a straight or branched C₂-C₆ alkoxy.

2. The compound of claim 1, wherein R₁ is at least a halogen.

3. The compound of claim 1, wherein R₁ is OH.

4. The compound of claim 3, wherein R₁ further comprises a halogen substitution.

5. The compound of claim 4, wherein the halogen substitution is a fluorine.

6. The compound of claim 1, wherein R₁ is OH in the para position.

7. The compound of claim 6, wherein R₁ further comprises a halogen substitution.

8. The compound of claim 7, wherein the halogen substitution is a fluorine.

9. The compound of claim 7, wherein the fluorine is in the meta position.

10. The compound of claim 1, wherein R₂ comprises a 3-, 4-, 5- or 6-membered alicyclic, heterocyclic or aromatic ring.

11. The compound of claim 10, wherein the ring of R₂ is alicyclic.

12. The compound of claim 11, wherein the alicyclic ring is cyclopropyl, cyclobutyl, cyclopentyl, cyclohexyl, cyclohexyl-2,3-ene, or 1-adamantyl.

13. The compound of claim 10, wherein the ring of R₂ is heterocyclic.

14. The compound of claim 13, wherein the ring of R₂ is para-hydroxymethylphenyl.

15. The compound of claim 13, wherein the heterocyclic ring is pyrimidine, pyridazine, pyrazine, pyridine, pyrazole, imidazole, pyrrole, pyran or furan.

16. The compound of claim 10, wherein the ring of R₂ is aromatic.

17. The compound of claim 16, wherein the aromatic ring is cyclobenzyl, 4-pyrimidyl, or 3-pyrimidyl.

18. The compound of claim 10, wherein the ring structure of R₂ comprises more than one ring.

19. The compound of claim 1, wherein the ring structure of R₂ is unsubstituted.

20. The compound of claim 1, wherein the ring structure of R₂ is substituted with at least one straight or branched C₁-C₆ alkyl, straight or branched C₁-C₆ alkenyl, straight or branched C₁-C₆ alkanoyl, straight or branched C₁-C₆ alkoxy, keto, carboxy, nitro, amino, hydroxy, halogen, cyano, diazo, thio, or hydroxyamino.

21. The compound of claim 20, wherein the ring structure of R₂ is substituted with at least one nitro, amino, hydroxyl or halogen.

22. The compound of claim 1, wherein R₃ is O.

23. The compound of claim 1, wherein R₄ is H.

24. The compound of claim 1 having formula I, wherein R₃ is O and R₄ is H.

25. The compound of claim 24, wherein R₁ is OH.

26. The compound of claim 25, wherein OH in the para position.

27. The compound of claim 24, wherein R₂ is cyclopropyl, cyclobutyl, cyclopentyl, cyclohexyl, cyclohexyl-2,3-ene, cyclobenzyl, 4-pyrimidyl, 3-pyrimidyl, 1-adamantyl, or methoxyphenyl.

28. The compound of claim 1, comprising or consisting of

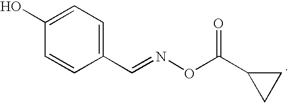

29. The compound of claim 1, comprising or consisting of

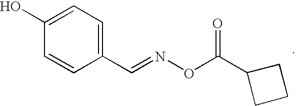

30. The compound of claim 1, comprising or consisting of

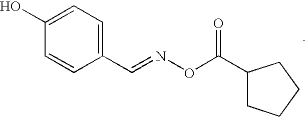

31. The compound of claim 1, comprising or consisting of

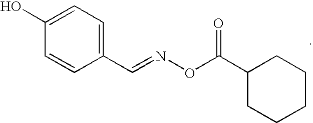

32. The compound of claim 1, comprising or consisting of

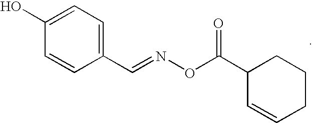

33. The compound of claim 1, comprising or consisting of

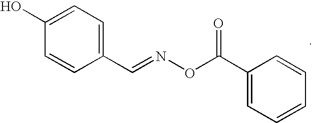

34. The compound of claim 1, comprising or consisting of

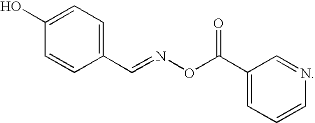

35. The compound of claim 1, comprising or consisting of

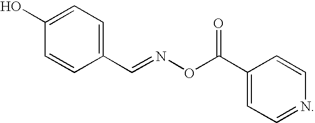

36. The compound of claim 1, comprising or consisting of

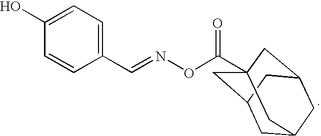

37. The compound of claim 1, comprising or consisting of

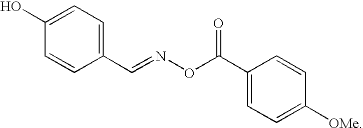

38. (canceled)

39. The compound of claim 38, comprising or consisting of

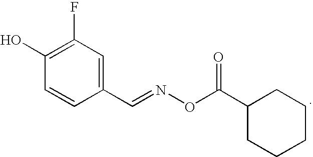

40. The compound of claim 38, comprising or consisting of

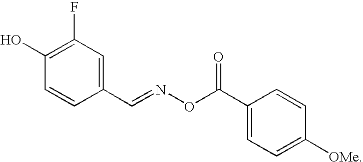

41. A pharmaceutical composition comprising the compound of claim 1, in a pharmaceutically acceptable excipient.

42-91. (canceled)

92. A method of inhibiting macrophage migration inhibitory factor (MIF) activity in a mammal, the method comprising administering the compound of claim 1 to the mammal in an amount effective to inhibit MIF activity in the mammal.

93-99. (canceled)

100. The method of claim 92, wherein the mammal has or is at risk for a condition that comprises an inflammatory cytokine cascade that is at least partially mediated by an MIF.

101. The method of claim 100, wherein the condition is cancer, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Retier's syndrome, or Hodgkins disease.

102. (canceled)

103. A method of treating or preventing inflammation in a mammal, the method comprising administering the compound of claim 1 to the mammal in an amount effective to treat or prevent the inflammation in the mammal.

104-110. (canceled)

111. The method of claim 103, wherein the mammal has cancer, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Retier's syndrome, or Hodgkins disease.

112-116. (canceled)

117. A method of treating a mammal having sepsis, septicemia, and/or endotoxic shock or at risk for sepsis, septicemia and/or endotoxic shock, the method comprising administering the compound of claim 1 to the mammal in an amount sufficient to treat or prevent the sepsis, septicemia and/or endotoxic shock.

118-125. (canceled)
